(19) United States
(12) Patent Application Publication
(10) Pub. No.: US 2015/0113741 A1
(43) Pub. Date: Apr. 30, 2015

(54) HAIR REMOVAL COMPOSITIONS

(71) Applicant: RECKITT & COLMAN (OVERSEAS) LIMITED, Berkshire (GB)

(72) Inventors: Tom Adams, Hull (GB); Catherine Colquhoun, Hull (GB); Carl Thompson, Hull (GB)

(21) Appl. No.: 14/395,662

(22) PCT Filed: Apr. 26, 2013
(86) PCT No.: PCT/GB2013/051065

§ 371 Date: Oct. 20, 2014

(30) Foreign Application Priority Data
Apr. 26, 2012 (GB) 1207499.3

(51) Int. Cl. A61K 8/19; A61Q 9/04

(52) U.S. Cl. CPC A61K 8/19; A61Q 9/04
USPC 8/161

(57) ABSTRACT

The present invention is directed to a hair removal composition which comprises an epilatory material or a depilatory active and a material having a thermal heat conductivity of from 50 to 2000 Wm⁻¹K⁻¹.

The present invention is directed to hair removal compositions. In particular, the present invention is directed to a heated epilatory or depilatory compositions that comprise a material which enables the compositions to reach a suitable temperature more quickly for better hair removal efficacy.

Currently, in order to achieve complete hair removal suitable compositions must be applied to a user's skin and are generally left for a period that can be up to 10 minutes prior to removal. In order to improve the speed of this process and thereby improve the experience of a user, such compositions are now commonly heated first. For example, both epilatory and depilatory compositions can be quite viscous and therefore heating them can make them significantly easier to apply.

According to a first aspect of the present invention there is provided a hair removal composition which comprises an epilatory material or a depilatory active and a material having a thermal heat conductivity of from 50 to 2000 Wm⁻¹K⁻¹.

The material can have a thermal heat conductivity of 200-400 Wm⁻¹K⁻¹. The material can have a thermal heat conductivity of 250-350 Wm⁻¹K⁻¹.

The material can be in the form of thermal interface material The thermal interface material can be selected from the group consisting of aluminium, aluminium-diamond, aluminium-graphite fibres, aluminium nitride, aluminium oxide, beryllium oxide, boron carbide, boron nitride, copper-graphite fibres, gold, gold coated powders, graphite, iron oxide, magnesium oxide, nickel gold additives, nickel graphite, nickel powders, silicon carbide, silicon nitride, silver, silver coated powders, silver-diamond, tin oxide, titanium carbide, titanium dioxide and zince oxide or combinations thereof. A preferred material is boron nitride.

The material having the thermal heat conductivity of up to 600 Wm⁻¹K⁻¹ can be added at a level of from 0.1 to 10% w/w. A preferred amount of said material is 3-7% w/w. A more preferred amount of said material is 4.5-5.5% w/w.

In an alternative preferred embodiment the material having the thermal heat conductivity of up to 600 Wm⁻¹K⁻¹ can be added at a level of from 0.05 to 1% w/w. A preferred amount of said material is 0.075-0.5% w/w. A more preferred amount of said material is 0.1% w/w.

The composition can be in the form of a cream, lotion or a wax. The wax can be in the form of a hot wax, a cold wax, and can include a sugar. If the composition is a depilatory composition then the composition can be in the form of a base cream, an aerosol cream or a shower cream.

When the composition is an epilatory composition, the epilatory material can be a resin in the form of a rosinous material, a hydrocarbon resin, a polylactic acid, a polyhydroxyalkanoate or can be a sugar-based material. The rosinous material may be, for example, rosin and/or a rosin based material. Preferably the rosin material is a rosin ester and/or colophony.

Typical examples of a rosin ester are glyceryl rosinate The hydrocarbon resin may be, for example, a terpene resin, a cyclic aliphatic hydrocarbon resin and/or a polycyclopentadiene. The sugar-based material can be a carbohydrate such as glucose syrup-based material, a sucrose syrup-based material or combinations thereof. The sugar-based material can also be a starch. A combination of sucrose and citric acid can also be used. One or more rosinous materials or hydrocarbon resins may be used. It is also possible to use a mixture of at least one rosinous material and at least one hydrocarbon resin.

The composition of the present invention desirably comprises at least 50 wt % of the rosinous material or hydrocarbon resin, preferably at least 60 wt %. Generally the composition comprises less than 90 wt % of the rosinous material or hydrocarbon resin, and preferably less than 80 wt %.

Preferably, the epilatory composition of the present invention further includes a particulate material in admixture with the matrix material.

Preferably the particulate material is a colloidal material. Preferably it has particles of mean diameter 1-200 nm, more preferably 5-100 nm, and most preferably 10-50 nm.

Preferably the particles are present in the epilatory material in an amount of at least 1% wt/wt, more preferably at least 2% wt/wt, and most preferably at least 3% wt/wt. Suitably they are present in an amount up to 40% wt/wt, preferably up to 20% wt/wt, and most preferably up to 10% wt/wt.

Preferred particulate materials for use in the present invention are siliceous materials. Especially preferred is fumed silica. Fumed silica typically comprises colloidal particles of mean diameter 1-200 nm. Preferably the fumed silica is of mean diameter 5-100 nm, more preferably 10-50 nm. The external surface area is typically in the range 15-380 m²/g. Fumed silicas are typically non-porous and thus have no internal surface area. They may be hydrophobic and of use in the present invention but preferred fumed silicas for use in the present invention are hydrophilic.

The epilatory composition may suitably comprise up to 40%, preferably up to 20%, of other components, which may include one or more of a natural wax, a fragrance, a polymer, a flexibiliser, an adhesion modifier, an essential oil, a silicone oil, a colorant, an anti-oxidant or a paraffin or mineral oil.

The composition can also comprise a polyethylene in the form of a homopolymer. The efficacy is especially improved when the epilatory formulation is in a cold wax strip format. It has also been observed to improve the stability of the wax on strips, and the resistance to flow under warm conditions. Preferably the polyethylene has a molecular weight from 100 to 1000, preferably from 250 to 800 more preferably from 300 to 600 unified mass units. This gives the advantage of ease of incorporation of the polyethylene into the hydrophobic particles of the invention by melting and blending. Polyethylene suitable for use in compositions of the invention is a substantially linear or non-branched polymer with the structure CH₃CH₂(CH₂CH₂)ₙCH₂ CH₃, where n is a mean number from 2 to 26, preferably from 5 to 15. Preferably at least 90% by weight of the polyethylene is linear or non-branched. A particularly preferred polyethylene is that sold under the registered trade name Performalene. Other suitable polymers include polybutene grades, ethylene and vinyl acetate, piperylene/butane/pentene/pentadiene coplolymer, goovean fibre viscose, however performalene is preferred.

When the composition is a depilatory composition, the depilatory composition may contain a skin-feel enhancing agent selected from at least one of silicone wax, talc and polyamide resin in the depilatory cream composition. In particular, the silicone wax, talc and/or a polyamide resin impart(s) a soft and velvety after-feel to the depilatory cream composition without affecting the composition's hair removal properties.

Talc is particularly preferred over other minerals which have previously been used in cosmetics as it confers a powdery after-feel which is desirable. In addition, it does not alter the colour of the composition. The talc may be present in an amount of 0.1 to 10 weight %, preferably 0.2 to 5 weight %, more preferably 0.5 to 3 weight %. In one embodiment, the composition includes 1 to 2 weight % talc.

The depilatory composition may further include a humectant. Suitable humectants include polyols, such as glycerine, propylene glycol and butylene glycol. Glycerine is preferred. The humectant may be present in an amount of 0 to 10 weight %, preferably 0.5 to 5 weight % .

The composition may comprise a polyamide resin as an alternative or in addition to the mineral. The polyamide resin may be present in an amount of 0.1 to 10 weight %, preferably 0.5 to 5 weight %, more preferably 1 to 3 weight %, for example 2 weight %. The polyamide resin is preferably Nylon-12.

The composition may also comprise a silicone wax as an alternative or in addition to the mineral and/or polyamide resin. Suitable silicone waxes include C₃₀-C₄₅ alkyl methicone and a silicone wax formed from stearoxytrimethylsilane and stearyl alcohol. The silicone wax is preferably C₃₀-C₄₅ alkyl methicone.

The silicone wax may be present in an amount of 0.1 to 10 weight %, preferably 0.5 to 5 weight %, more preferably 1 to 3 weight %, for example 1 to 2 weight %.

The emollient is selected from at least one of mineral oil, silicone and emollient esters. Together with the silicone wax, mineral and/or polyamide resin (and optional humectant), the emollient plays an important role in providing the depilatory cream composition with its desired skin-feel characteristics.

The emollient may be present in an amount of 1 to 10 weight %, preferably 3 to 7 weight % of the composition.

Mineral oil may be present in an amount of 0 to 10 weight %, preferably 0.5 to 5 weight % of the composition.

Silicone oil may be present in an amount of 0 to 10 weight %, preferably 0.5 to 5 weight %, for example 1 to 4 weight % of the composition.

Emollient esters may be present in an amount of 0 to 10 weight %, preferably 0.5 to 5 weight % of the composition, for example 1 to 3 weight %.

It is possible for the emollient to consist essentially of mineral oil. For example, in one embodiment, the composition includes talc and an emollient that consists essentially of mineral oil. In this embodiment, the emollient is present in an amount of 3 to 6 weight %, preferably 5 weight %. The talc is present in an amount of 0.3 to 1 weight %, preferably 0.5 weight %. Where an emollient consisting essentially of mineral oil emollient is employed, the composition preferably includes a humectant, such as glycerine.

It is also possible for the emollient to comprise or consist essentially of silicone oil(s). Preferably, a combination of silicone oils are present. The silicone oil may include at least one of cyclopentasiloxane, dimethiconol and dimethicone. Preferably, the silicone oil comprises cyclopentasiloxane, dimethiconol and dimethicone. The silicone oil may include 0.1 to 5 weight %, preferably 1 to 2 weight % dimethicone; and/or to 5 weight %, for example, 1 to 3 weight % cyclopentasiloxane and dimethiconol.

It is possible for the emollient to consist essentially of an emollient ester. However, the emollient ester is preferably used in combination with a mineral oil and/or a silicone oil.

In one embodiment, the emollient comprises at least two of mineral oil, silicone oil and emollient esters. For example, the emollient may include mineral oil and silicone oil, or mineral oil and emollient esters, or silicone oil and emollient esters. In one embodiment, the emollient includes mineral oil, silicone oil and emollient esters.

Any suitable silicone oil may be employed. Examples include cyclopentasiloxane, dimethiconol and dimethicone. The total amount of silicone oil in the composition may be 0.1 to 10 weight %, for example, 2 to 5 weight %.

Any suitable emollient ester may be employed. Suitable examples include isopropyl palmitate, isopropyl myristate, myristyl lactate, cetyl esters, isotridecyl isononanoate, C₁₂₋₁₅ alkyl benzoate, caprylic/capric triglyceride and pentaerythrityl tetraisostearate.

In one embodiment, the emollient comprises mineral oil. The mineral oil may be present in an amount of 3 to 6 weight %, preferably 5 weight %. In a preferred embodiment, this combination of emollients is used together with at least one of talc and polyamide resin.

The depilatory active is a compound capable of degrading keratin and may be, for example, a sulphur compound such as potassium thioglycolate, dithioetythritol, thioglycerol, thioglycol, thioxanthine, thipsalicylcic acid, N-acetyl-L-cysteine, lipic acid, NaHSO₃, Li₂S, Na₂S, K₂S, MgS, CaS, SrS, BaS, (NH₄)₂S, sodium dihydrolipoate 6, 8-dithiooctanoate, sodium 6,8-dithiooctanoate, salts of hydrogen sulphide for example NaSH or KSH, thioglycolic acid, thioglycerol, 2-mercaptopropionic acid, 3-mercaptropropionic acid, thiomalic acid, ammonium thioglycolate, glyceryl monothioglycolate, monoethanolamine thioglycolate, monoethanolamine, thioglycolic acid, diammonium dithiodiglycolate, ammonium thiolactate, monoethanolamine thiolactate, thioglycolamide, homo-cysteine, cysteine, glutathione, dithiothreitol, dihydrolipoic acid, 1,3-dithiopropanol, thioglycolamide, glycerylmonothioglycolate, thioglycolhydrazine, keratinase, hydrazine sulphate, hydrazine disulphate triisocyanate, guanidine thioglycolate, calcium thioglycolate and/or cysteamine. However, the composition is preferably substantially or, more preferably, is completely free from depilatory agents that destroy the thermodynamic equilibrium or the surface tension of the composition; examples of such agents include alkali metal sulphides.

Preferred depilatory compounds are thioglycolates, or their precursor thioglycolic acid. Most preferred is potassium thioglycolate, which may be produced by mixing thioglycolic acid with a neutralising source of potassium hydroxide (as noted above excess potassium hydroxide over that required to effect neutralisation cannot be used).

The depilatory active may be present in an amount of 2 to 25 weight %, preferably 5 to 20 weight %, more preferably 10 to 15 weight %. In one embodiment, the composition includes potassium thioglycolate in an amount of 2 to 25 weight %, preferably 5 to 20 weight %, more preferably 10 to 15 weight %.

The depilatory composition of the present invention preferably includes water. Water may be present in an amount of at least 40 weight %, preferably at least 50 weight %. Suitable amounts of water range from 40 to 70 weight %, preferably 50 to 65 weight %, more preferably 55 to 60 weight %.

The depilatory composition may optionally include one or more surfactant(s). The surfactant may be anionic, cationic or non-ionic. It is preferably non-ionic. Examples of suitable surfactants include cetearyl phosphate, cetearyl alcohol, cetearyl glucoside, cetostearyl alcohol and/or ceteareth 20. It is preferably present in an amount of from 0.5 to 15 wt % relative to the weight of the depilatory composition, more preferably from 1 to 10 wt %.

The depilatory composition may optionally include a source of alkalinity. This may include hydroxides, such as hydroxides of alkali and alkaline earth metals. Suitable hydroxides include sodium hydroxide, potassium hydroxide, calcium hydroxide and magnesium hydroxide. Preferably, calcium hydroxide is employed, optionally together with potassium hydroxide. The source of alkalinity (e.g. calcium hydroxide) may be present in an amount of 0.1 to 10 weight %, preferably 1 to 6 weight %, for example 2 to 5 weight % of the depilatory cream composition.

The depilatory composition preferably has a pH of greater than 7, for example, 9 to 12.5.

Optionally, the composition includes an accelerator that will accelerate the hair removal reaction. Examples of such accelerators include urea, thiourea, dimethyl, isosorbide (DMI), ethoxydiglycol (Transcutol) or methyl propyl diol (MP diol). Preferably the accelerator is urea. The composition according to the invention preferably comprises from 5% to 15% wt, more preferably from 6 to 10 wt % of an accelerator (e.g. urea).

The depilatory composition desirably includes a chelating agent, such as sodium gluconate. The chelating agent may be present in an amount of less than 1 weight %, preferably 0.01 to 0.5 weight %, for example 0.05 to 0.1 weight %.

The depilatory cream composition may also include an additive that prevents phase separation. Suitable additives include polymers or copolymers of acrylic acid, for example, an acrylate copolymer. Such additives may be present in an amount of up to 2 weight %, preferably less than 1 weight %, more preferably less than 0.5 weight %, for example 0.1 to 0.4 weight %.

The epilatory or depilatory composition may comprise other optional ingredients, such as pigments and fillers, such as clays or talcs. Examples of suitable clays include sodium magnesium silicate, magnesium trisilicate and titanium dioxide. The inclusion of a clay, preferably sodium magnesium silicate, more preferably in an amount of from 0.1 to 10 wt % relative to the weight of the depilatory composition, most preferably from 0.1 to 1 wt %, is particularly advantageous, since this provides sodium and magnesium ions for the buffer system and improves the efficiency of depilation.

Optionally, additives such as aloe vera and Vitamin E may also be included in the epilatory or depilatory composition. Such additives are employed in amounts of less than 1 weight %, for example, 0.1 to 0.5 weight % of the composition.

According to a second aspect of the present invention there is provided the use of a material having a thermal heat conductivity of from 50-2000 Wm⁻¹K⁻¹ to reduce the heat capacity of a hair removal composition.

The material can have a thermal heat conductivity of 200-400 Wm⁻¹K⁻¹. The material can have a thermal heat conductivity of 250-350 Wm⁻¹K⁻¹.

The material can be in the form of thermal interface material. The thermal interface material can be selected from the group consisting of aluminium, aluminium-diamond, aluminium-graphite fibres, aluminium nitride, aluminium oxide, beryllium oxide, boron carbide, boron nitride, copper-graphite fibres, gold, gold coated powders, graphite, iron oxide, magnesium oxide, nickel gold additives, nickel graphite, nickel powders, silicon carbide, silicon nitride, silver, silver coated powders, silver-diamond, tin oxide, titanium carbide, titanium dioxide and zince oxide or combinations thereof. A preferred material is boron nitride.

The material having the thermal heat conductivity of up to 600 Wm⁻¹K⁻¹ can be added at a level of from 0.1 to 10% w/w. A preferred amount of said material is 3-7% w/w. A more preferred amount of said material is 4.5-5.5% w/w.

In an alternative preferred embodiment the material having the thermal heat conductivity of up to 600 Wm⁻¹K⁻¹ can be added at a level of from 0.05 to 1% w/w. A preferred amount of said material is 0.075-0.5% w/w. A more preferred amount of said material is 0.1% w/w.

The hair removal composition can be the same as that described in respect of the first aspect of the invention.

According to a third aspect of the present invention there is provided the use of a material having a thermal heat conductivity of from 50-2000 Wm⁻¹K¹ to increase the thermal conductivity of a hair removal composition.

The material can have a thermal heat conductivity of 200-400 Wm⁻¹K⁻¹. The material can have a thermal heat conductivity of 250-350 Wm⁻¹K⁻¹.

The material can be in the form of thermal interface material. The thermal interface material can be selected from the group consisting of aluminium, aluminium-diamond, aluminium-graphite fibres, aluminium nitride, aluminium oxide, beryllium oxide, boron carbide, boron nitride, copper-graphite fibres, gold, gold coated powders, graphite, iron oxide, magnesium oxide, nickel gold additives, nickel graphite, nickel powders, silicon carbide, silicon nitride, silver, silver coated powders, silver-diamond, tin oxide, titanium carbide, titanium dioxide and zinc oxide or combinations thereof. A preferred material is boron nitride.

The material having the thermal heat conductivity of up to 600 Wm⁻¹K⁻¹ can be added at a level of from 0.1 to 10% w/w. A preferred amount of said material is 3-7% w/w. A more preferred amount of said material is 4.5-5.5% w/w.

In an alternative preferred embodiment the material having the thermal heat conductivity of up to 600 Wm⁻¹K⁻¹ can be added at a level of from 0.05 to 1% w/w. A preferred amount of said material is 0.075-0.5% w/w. A more preferred amount of said material is 0.1% w/w.

Typically the material having a thermal heat conductivity of from 50-2000 Wm⁻¹K⁻¹ increases the conductivity of the composition by at least 10%. Preferably the increase in conductivity is at least 25%.

The hair removal composition can be the same as that described in respect of the first aspect of the invention.

According to a fourth aspect of the present invention there is provided a hair removal composition which comprises an epilatory material or a depilatory active and a ceramic material wherein the hair removal composition has a heat capacitance of 1.2-1.70 J/(g° C.).

The ceramic material can be in the form of thermal interface material. The thermal interface material can be selected from the group consisting of aluminium, aluminium-diamond, aluminium-graphite fibres, aluminium nitride, aluminium oxide, beryllium oxide, boron carbide, boron nitride, copper-graphite fibres, gold, gold coated powders, graphite, iron oxide, magnesium oxide, nickel gold additives, nickel graphite, nickel powders, silicon carbide, silicon nitride, silver, silver coated powders, silver-diamond, tin oxide, titanium carbide, titanium dioxide and zinc oxide or combinations thereof. A preferred material is boron nitride.

The ceramic material can be added at a level of from 0.1 to 10% w/w. A preferred amount of said material is 3-7% w/w. A more preferred amount of said material is 4.5-5.5% w/w.

In an alternative preferred embodiment the ceramic material can be added at a level of from 0.05 to 1% w/w. A preferred amount of said material is 0.075-0.5% w/w. A more preferred amount of said material is 0.1% w/w.

Typically the ceramic material reduces the heat capacitance of the composition by at least 10%. Preferably the reduction in heat capacitance is between 10% and 20%.

The hair removal composition can be the same as that described in respect of the first aspect of the invention.

According to a fifth aspect of the present invention there is provided the use of a composition as described in the first or fourth aspects of the present invention for the removal of hair from the skin of an individual.

According to a sixth aspect of the present invention there is provided a method of removing hair from the skin of an individual comprising the steps of (a) heating a composition as described in either the first or fourth aspects of the present invention; (b) applying said heated composition to the skin of an individual; and (c) removing said composition after a time sufficient to allow for removal of the hair.

Typically the residence time of the composition on the skin is less than 10 minutes, more preferably not more than 6 minutes. Very suitably the residence time is 1 to minutes, about 2 to 3 minutes being especially preferred.

For the purposes of the present invention the use of the term ‘cold wax strip’ is intended to cover materials that are warmed manually by a consumer prior to application to the skin.

The present invention will now be described in more detail with reference to the accompanying Figures in which:

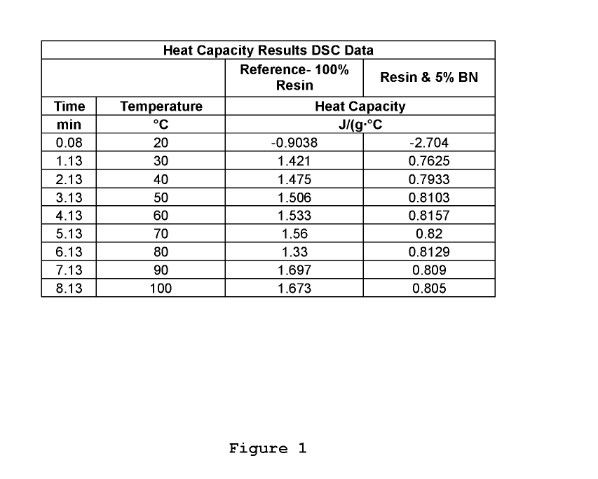

FIG. 1 illustrates the effect of the inclusion of 5% boron nitride on the heat capacity of an epilatory composition as measured by DSC;

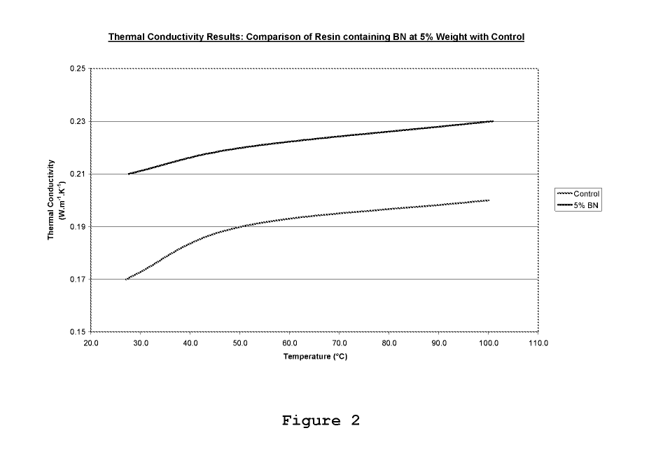

FIG. 2 illustrates the effect of adding boron nitride to a standard epilatory composition on the thermal conductivity as measured by Guarded Heat Flow;

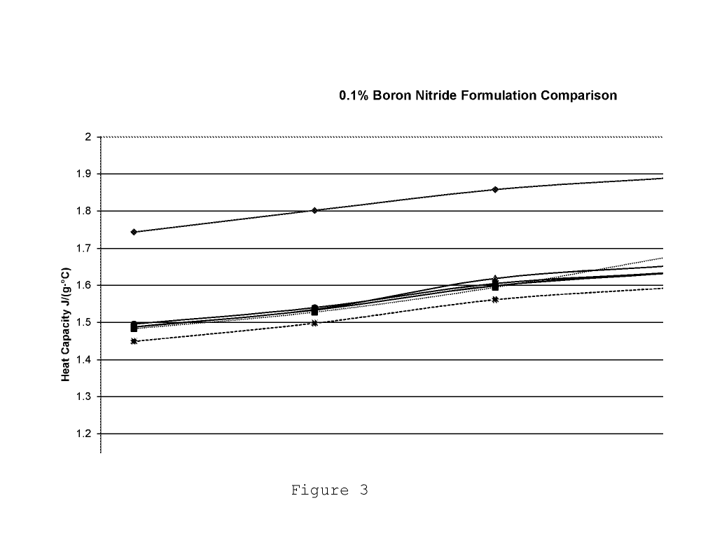

FIG. 3 illustrates the effect of the inclusion of 0.1% boron nitride on the heat capacity of an epilatory composition as measured by differential scanning calorimetry (DSC); and

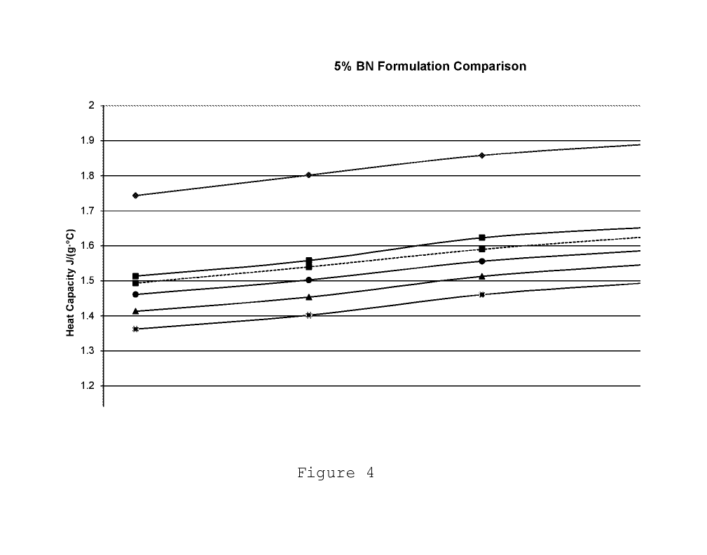

FIG. 4 illustrates further examples of the effect of the inclusion of 5% boron nitride on the heat capacity of an epilatory composition as measured by differential scanning calorimetry (DSC).

Example embodiments of the invention are shown in the table below:

| Standard Name | Ex 1 (%) | Ex 2 (%) | Ex 3 (%) |
|---|---|---|---|
| Rosin Ester Mix 3 | 98.486 | 98.086 | 93.586 |
| Boron Nitride | 0.10 | 0.50 | 5.00 |
| Cosmetic ingredient | 0.10 | 0.10 | 0.10 |
| Polyethylene | 1.00 | 1.00 | 1.00 |
| Covalim | 0.30 | 0.30 | 0.30 |
| Dye | 0.014 | 0.014 | 0.014 |

The compositions can be formulated using standard techniques known to the man skilled in the art.

FIG. 1 illustrates that when boron nitride is added to an epilatory composition the heat capacity of the composition is lowered such that the composition can be heated to the desired temperature of use more quickly than standard compositions.

FIG. 2 illustrates the effect of adding boron nitride to a standard epilatory composition.

FIGS. 3 and 4 illustrate the effect of adding boron nitride having different crystal structures to a standard epilatory composition. It can be seen that there is no significant difference between the different forms of boron nitride, such as single crystal platelet and hexagonal crystalline.

An advantage of the present invention is that there is provided an epilatory wax or depilatory cream that reaches its temperature of use more quickly thus making the process of hair removal faster as well as providing better coverage/wetting of hair. In addition, less energy is required for the composition to reach its temperature of use with the concomitant benefits this brings. Additional advantages of the pre-warmed composition are based around the sensory feel it provides to the consumer.

Further modifications and developments can be made without departing form the scope of the invention described herein.

1. A hair removal composition comprising:
one of an epilatory material and a depilatory active; and
a material having a thermal heat conductivity of from 50 to 2000 Wm⁻¹K⁻¹.

2. The hair removal composition as claimed in claim 1, wherein the material has a thermal heat conductivity of from 200 to 400 Wm⁻¹K⁻¹.

3. The hair removal composition as claimed in claim 1, wherein the material is in the form of a thermal interface material selected from the group consisting of aluminium, aluminium-diamond, aluminium-graphite fibres, aluminium nitride, aluminium oxide, beryllium oxide, boron carbide, boron nitride, copper-graphite fibres, gold, gold coated powders, graphite, iron oxide, magnesium oxide, nickel gold additives, nickel graphite, nickel powders, silicon carbide, silicon nitride, silver, silver coated powders, silver-diamond, tin oxide, titanium carbide, titanium dioxide and zinc oxide or combinations thereof.

4. The hair removal composition as claimed in claim 1, wherein the material is boron nitride.

5. The hair removal composition as claimed in claim 1, wherein the material has a thermal heat conductivity of up to 600 Wm⁻¹K⁻¹ at a level of from 0.1 to 10% w/w.

6. The hair removal composition as claimed in claim 5, wherein the material is at a level of from 3-7% w/w.

7. The hair removal composition as claimed in claim 5, wherein the material is at a level of from 4.5-5.5% w/w.

8. The hair removal composition as claimed in claim 1, wherein the material is at a level of from 0.05 to 1% w/w.

9. The hair removal composition as claimed in claim 8, wherein the material is at a level of from 0.075-0.5% w/w.

10. The hair removal composition as claimed in claim 8, wherein the material is at a level of 0.1% w/w.

11-20. (canceled)

21. A hair removal composition comprising:
one of an epilatory material and a depilatory active; and
a ceramic material;
wherein the hair removal composition has a heat capacitance of 1.2-1.70 J/(g° C.).

22. The hair removal composition as claimed in claim 21, wherein the ceramic material is in the form of thermal interface material.

23. The hair removal composition as claimed in claim 22, wherein the thermal interface material is selected from the group consisting of aluminium, aluminium-diamond, aluminium-graphite fibres, aluminium nitride, aluminium oxide, beryllium oxide, boron carbide, boron nitride, copper-graphite fibres, gold, gold coated powders, graphite, iron oxide, magnesium oxide, nickel gold additives, nickel graphite, nickel powders, silicon carbide, silicon nitride, silver, silver coated powders, silver-diamond, tin oxide, titanium carbide, titanium dioxide and zinc oxide or combinations thereof.

24. The hair removal composition as claimed in claim 22, wherein the ceramic material is boron nitride.

25. The hair removal composition as claimed in claim 21, wherein the ceramic material is at a level of from 0.1 to 10% w/w.

26. The hair removal composition as claimed in claim 25, wherein the amount of the ceramic material is from 3-7% w/w.

27. The hair removal composition as claimed in claim 25, wherein the amount of the ceramic material is from 4.5-5.5% w/w.

28. The hair removal composition as claimed in claim 21, wherein the ceramic material is at a level of from 0.05 to 1% w/w.

29. The hair removal composition as claimed in claim 28, wherein the amount of the ceramic material is from 0.075-0.5% w/w.

30. The hair removal composition as claimed in claim 28, wherein the amount of the ceramic material is 0.1% w/w.

31. The hair removal composition as claimed in claim 21, wherein the ceramic material reduces the heat capacitance of the composition by at least 10% without the ceramic material.

32. The hair removal composition as claimed in claim 31, wherein the reduction in heat capacitance is between 10% and 20%.

33. (canceled)

34. A method of removing hair from skin of an individual comprising:
heating the composition as claimed in claim 1;
applying the heated composition to skin of an individual; and
removing the composition after a time sufficient to allow for removal of hair.

35. The method as claimed in claim 34, wherein the residence time of the composition on the skin is less than 10 minutes.

36. The method as claimed in claim 34, wherein the residence time of the composition on the skin is not more than 6 minutes.

37. The method as claimed in claim 34, wherein the residence time of the composition on the skin is from 1 to 5 minutes.

38. The method as claimed in claim 34, wherein the residence time of the composition on the skin is from 2 to 3 minutes.
